(19) United States
(12) Patent Application Publication
(10) Pub. No.: US 2020/0368272 A1
(43) Pub. Date: Nov. 26, 2020

(54) COMPOSITION FOR AMELIORATING POST-CEREBRAL STROKE SEQUELA

(71) Applicant: MiZ Company Limited, Kanagawa (JP)

(72) Inventors: Fumitake Satoh, Kanagawa (JP); Shinichi Hirano, Kanagawa (JP); Yusuke Ichikawa, Kanagawa (JP)

(21) Appl. No.: 16/849,057

(22) Filed: Apr. 15, 2020

(30) Foreign Application Priority Data
Apr. 16, 2019 (JP) 2019-077629

(51) Int. Cl. A61K 33/00; A61K 47/02; A61P 25/04

(52) U.S. Cl. CPC A61K 33/00; A61P 25/04; A61K 47/02

(57) ABSTRACT

This application provides a composition for ameliorating a post-cerebral stroke sequela in a human, comprising molecular hydrogen as an active ingredient, and a method for ameliorating a post-cerebral stroke sequela, comprising administering the composition to a human having a post-cerebral stroke sequela.

### CROSS-REFERENCE TO RELATED APPLICATION

This application claims the benefit of JP Patent Application No. 2019-077629, filed on Apr. 16, 2019, which is incorporated by reference herein in its entirety.

### FIELD OF THE INVENTION

The present invention relates to a composition for ameliorating a post-cerebral stroke sequela in a human, comprising molecular hydrogen as an active ingredient.

The present invention also relates to a method for ameliorating a post-cerebral stroke sequela, comprising administering the composition to a human having a post-cerebral stroke sequela.

### BACKGROUND ART

Cerebral vascular diseases such as cerebral stroke tend to be accompanied by severe sequelae due to their onset, and are diseases having a higher percentage of causing a patient to become bedridden. In the case of cerebral stroke, it is known that severe sequelae, such as spasticity, pain, paralysis, language disorders, and lower body paralysis, typically occur. For example, central post-stroke pain is classified as a neuropathic pain, and significantly impairs daily life as even mild stimuli induce pain. Although treatment by drugs is used, no effective treatment method has been established (S. Harada et al., Japanese Journal of Pharmaceutical Palliative Care and Sciences, 2013; 6: 51-56).

In addition to chemotherapy, rehabilitation with physiotherapists to recover motor function, training with speech therapists to recover language function, and the like are usually performed for the sequelae, but complete remission is often difficult.

The following have been reported on hydrogen treatments for cerebral stroke patients.

JP 2017-210433 A discloses a therapeutic agent for acute cerebral infarction containing molecular hydrogen as an active ingredient for ameliorating the efficiency of rehabilitation after treatment, wherein the acute cerebral infarction is a cerebral infarction within 24 hours after onset, and the concentration of molecular hydrogen is, for example, 1 to 4% (V/V).

H. Ono et al. (J Stroke and Cerebrovascular Diseases 2017; 26 (11): 2587-2594) disclose that a safe and effective hydrogen treatment is given to patients with acute cerebral infarction to protect against acute oxidative stress.

H. Ono et al. (Medical Gas Research 2011; 1:12) disclose a combination therapy by administering Edaravon, a hydroxyl radical scavenger, and molecular hydrogen to patients with acute brainstem infarction.

Although all of these reports relate to the usefulness of hydrogen therapy for patients with acute cerebral infarction, the effects of applying hydrogen therapy to reduce or improve post-cerebral stroke sequelae are not clear.

### SUMMARY OF THE INVENTION

An object of the present invention is to provide a composition, as well as a method, for ameliorating a post-cerebral stroke sequela in humans.

The present inventors have now unexpectedly found that molecular hydrogen is effective for achievement of the above object. Specifically, the present invention includes the following features.
(1) A composition for ameliorating a post-cerebral stroke sequela in a human, comprising molecular hydrogen as an active ingredient.
(2) The composition according to (1), wherein the cerebral stroke is selected from the group consisting of cerebral hemorrhage (also referred to as “intracerebral hemorrhage”), subarachnoid hemorrhage, and cerebral infarction (e.g., atherothrombotic cerebral infarction, lacunar infarction, cardiogenic cerebral embolism, and cerebral ischemia).
(3) The composition according to (1) or (2), wherein the sequela is at least one symptom selected from the group consisting of movement disorders, sensory disturbances, language disorders, central post-stroke pain, and allodynia.
(4) The composition according to any one of (1) to (3), wherein the composition is a gas comprising an effective amount of molecular hydrogen and air or oxygen.
(5) The composition according to (4), which is a gas comprising molecular hydrogen in an amount greater than zero (0) and not greater than 18.5 vol %.
(6) The composition according to any one of (1) to (3), wherein the composition is a biocompatible liquid comprising an effective amount of molecular hydrogen.
(7) The composition according to (6), wherein the biocompatible liquid is water, physiological saline, or a transfusion.
(8) The composition according to (6) or (7), wherein a concentration of molecular hydrogen dissolved in the biocompatible liquid is 1 to 10 ppm.
(9) A method for ameliorating a post-cerebral stroke sequela in a human, comprising administering the composition according to any one of (1) to (8) comprising molecular hydrogen as an active ingredient to a human having a post-cerebral stroke sequela.

According to the present invention, post-cerebral stroke sequelae (e.g., pain, bodily unsteadiness, spasticity, numbness of the limbs, and language disorders) can be improved (or reduced or relieved) in humans by the administration of molecular hydrogen.

### BRIEF DESCRIPTION OF THE DRAWINGS

FIG. 1 shows the improvement of pain, which is a post-cerebral stroke sequela of a human having suffered from cerebral stroke (pontine hemorrhage), by inhalation of air containing molecular hydrogen. The values indicate the mean±standard deviation of VAS during the period of about 15 days, and the P value indicates a statistically significant difference from the start of inhalation of air containing molecular hydrogen (0.5 month). Moreover, the VAS on the vertical axis represents the value of Visual Analogue Scale (scale: 0 to 5), with VAS “0” indicating a condition with none of the above symptoms, and VAS “5” indicating a condition in which each of the above symptoms is the most severe;

FIG. 2 shows the improvement of bodily unsteadiness, which is a post-cerebral stroke sequela in a human suffering from cerebral stroke (pontine hemorrhage), by inhalation of air containing molecular hydrogen. The values indicate the mean±standard deviation of VAS during the period of about 15 days, and the P value indicates a statistically significant difference from the start of inhalation of air containing molecular hydrogen (0.5 month). Moreover, the VAS on the vertical axis represents the value of Visual Analogue Scale (scale: 0 to 5), with VAS “0” indicating a condition with none of the above symptoms, and VAS “5” indicating a condition in which each of the above symptoms is the most severe; and

FIG. 3 shows the improvement of numbness of the limbs, which is a post-cerebral stroke sequela of a human suffering from cerebral stroke (pontine hemorrhage), by inhalation of air containing molecular hydrogen. The values indicate the mean±standard deviation of VAS during the period of about 15 days, and the P value indicates a statistically significant difference from the start of inhalation of air containing molecular hydrogen (0.5 month). Moreover, the VAS on the vertical axis represents the value of Visual Analogue Scale (scale: 0 to 5), with VAS “0” indicating a condition with none of the above symptoms, and VAS “5” indicating a condition in which each of the above symptoms is the most severe.

### DETAILED DESCRIPTION

The present invention is further described in detail.

### 1. Composition Containing Molecular Hydrogen

The composition of the present invention can improve a post-cerebral stroke sequela in humans. The active ingredient is molecular hydrogen.

As used herein, the “human”, which is a subject, includes those who are receiving treatment for cerebral stroke and those who have had cerebral stroke and have a post-cerebral stroke sequela.

As used herein, the term “molecular hydrogen” may be used interchangeably with hydrogen molecule, gaseous hydrogen, or hydrogen gas. In addition, as described later, the composition comprising molecular hydrogen may be a gas mixture of a predetermined amount of molecular hydrogen and a predetermined amount of, for example, air or oxygen, or the composition may be a biocompatible liquid in which a predetermined amount of molecular hydrogen is dissolved.

As used herein, the term “ cerebral stroke” includes those in which the brain blood vessels rupture and hemorrhage, for example, cerebral hemorrhage or subarachnoid hemorrhage, as well as those in which the blood vessels are clogged, and blood is prevented to flow in the brain, for example, cerebral infarction (e.g., atherothrombotic cerebral infarction, lacunar infarction, cardiogenic cerebral embolism, cerebral ischemia, or the like).

In the cerebral hemorrhage, a mass of blood resulting from bleeding in the brain compresses and destroys brain cells, causing various symptoms, such as headache, motor paralysis, language disorders, and decline in consciousness. The regions prone to cerebral hemorrhage are known, and are mainly classified according to the names of the regions: putaminal hemorrhage, thalamic hemorrhage, subcortical hemorrhage, pontine hemorrhage, cerebellar hemorrhage, and the like.

Subarachnoid hemorrhage occurs when a swelling formed at the bifurcation of blood vessels outside the brain ruptures and hemorrhages in the space between the brain and the subarachnoid.

In cerebral infarction, a transient cerebral ischemic attack may occur as a precursor to its onset, and it is said that a severe cerebral infarction tends to occur if left untreated without appropriate treatment. When blood vessels are clogged, the blood flow does not reach the brain cells beyond the clogging, and oxygen and nutrients can no longer be delivered, which causes damage (cerebral ischemia). Examples of cerebral infarction include lacunar infarction (the arteries in the brain become thinner, which clogs the blood vessels), atherothrombotic cerebral infarction (caused by atherosclerosis, blood coagulates on the spot and causes infarction), and cardiogenic cerebral embolism (a blood clot formed in the heart due to arrhythmia clogs blood vessels in the brain and causes infarction).

As used herein, the term “post-cerebral stroke sequela” refers to a disorder that remains in the brain or body even after performing appropriate treatment for the cerebral stroke attack. The types and severity of the disorder vary depending on the regions and severity of the hemorrhage or infarction in the brain. Examples of the sequela include movement disorders (e.g., hemiplegia, unilateral paralysis, deglutition, spasticity, bodily unsteadiness, and the like), sensory disturbances (e.g., numbness of the limbs, insensitivity, and the like), language disorders (e.g., dysarthria, aphasia, and the like), high-order functional disorders (e.g., cognitive impairment, memory impairment, social behavior impairment, attention disorder, and the like), central post-stroke pain (CPSP), and allodynia.

As used herein, the “central post-stroke pain” refers to pain that occurs several weeks to several months after the brain was damaged by cerebral stroke. Moreover, such pain also includes allodynia, in which pain is experienced in response to normally painless stimuli (for example, numbness of the limbs).

In the Examples described later, a trend toward improvement was observed after about 1 month for central post-stroke pain, after about 1 to 3.5 months for bodily unsteadiness, spasticity, and language disorder, and after about 4.5 months for numbness of the limbs, as a result of continuous administration of the composition of the present invention.

Spasticity is a movement disorder, and symptoms include muscles that are too tense, difficulty to move the limbs, involuntary movements, bending of the elbow, and toes bending toward the back of the feet.

As used herein, the “hydrogen”, which is the active ingredient of the composition of the present invention, is molecular hydrogen. Moreover, the term “molecular hydrogen” as used herein refers to, in molecular formula, H₂, D₂ (deuterium), HD (hydrogen deuteride), or a gas mixture thereof. D₂ is costly, but is known to have a stronger superoxide scavenging effect than H₂. The hydrogen usable in the present invention is H₂, D₂ (deuterium), HD (hydrogen deuteride), or a gas mixture thereof, preferably H₂, or, instead of H₂ or mixed with H₂, D₂ and/or HD may be used.

The molecular hydrogen-containing gas is preferably air containing molecular hydrogen or a gas mixture containing molecular hydrogen and oxygen. The concentration of molecular hydrogen in the molecular hydrogen-containing gas is greater than zero (0) and not greater than 18.5 vol %, for example, 0.5 to 18.5 vol %, preferably 1 to 10 vol %, such as 2 to 8 vol %, 3 to 7 vol %, 3 to 6 vol %, 4 to 6 vol %, 4 to 5 vol %, 5 to 10 vol %, 5 to 8 vol %, 6 to 8 vol %, and 6 to 7 vol %, more preferably 5 to 8 vol %, such as 6 to 8 vol % and 6 to 7 vol %. In the present invention, while remaining equal to or below the explosion limit, the higher the molecular hydrogen concentration is, the greater the effect of ameliorating (or reducing) a post-cerebral stroke sequela tends to be.

In this context, with regard to generally employed hydrogen gas inhalation therapy, the hydrogen gas exhibits an effect to improve a disease (e.g., cancer) at a high concentration of not less than about 65 vol %, whereas in the present invention, the hydrogen containing composition is preferably administered to subjects (e.g. humans) under safe conditions and thus, by using a low hydrogen concentration greater than zero (0) and not greater than 18.5%, it is possible to exhibit an effect of sufficiently ameliorating post-cerebral stroke sequelae. In addition, since molecular hydrogen is a flammable and explosive gas, it is preferable to administer the hydrogen to humans by incorporating hydrogen in the composition of the present invention under safe conditions for ameliorating (or reducing) a post-cerebral stroke sequela.

When the gas other than molecular hydrogen is air, the concentration of air is, for example, in the range of 81.5 to 99.5 vol %.

When the gas other than molecular hydrogen is a gas containing oxygen, the concentration of oxygen is, for example, in the range of 21 to 99.5 vol %.

As other main gases, nitrogen may be contained, for example.

The molecular hydrogen-dissolved liquid is, specifically, an aqueous liquid in which molecular hydrogen is dissolved, wherein the aqueous liquid includes, but is not limited to, for example, water (for example, purified water, sterilized water), physiological saline, buffered physiological saline (for example, pH 4 to 7.4), infusions, transfusions, injection solutions, beverages (for example, tea drinks such as green tea and black tea, fruit juices, green juices, vegetable juices, and the like). The molecular hydrogen concentration in the molecular hydrogen-dissolved liquid includes, but is not limited to, for example, 1 to 10 ppm, preferably 1.2 to 8 ppm, such as 1.5 to 7 ppm, 1.5 to 5 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 5 to 10 ppm, 5 to 9 ppm, and 5 to 8 ppm, and more preferably 3 to 8 ppm, such as 3 to 7 ppm, 4 to 8 ppm, and 5 to 8 ppm.

A medicament for treating a post-cerebral stroke sequela may be added to the molecular hydrogen-dissolved liquid. Alternatively, the medicament may be administered separately from the administration of the molecular hydrogen-dissolved liquid or molecular hydrogen-containing gas.

The molecular hydrogen-containing gas or the molecular hydrogen-dissolved liquid is blended to achieve a predetermined molecular hydrogen concentration, then is filled, for example, in a pressure-resistant container (for example, a stainless steel cylinder, an aluminum can, preferably a pressure-resistant plastic bottle (for example, a pressure-resistant PET bottle) and a plastic bag, and an aluminum bag, which are preferably laminated with aluminum film on the inside). Aluminum has almost no property of allowing hydrogen molecules to permeate. Alternatively, at the time of administration, the molecular hydrogen-containing gas or the molecular hydrogen-dissolved liquid may be produced in situ using an apparatus such as a hydrogen gas generator, a hydrogen water generator, or a hydrogen gas-adding apparatus, for example, a known or commercially available hydrogen gas supplying apparatus (i.e., an apparatus for generating molecular hydrogen-containing gas), a hydrogen-adding equipment (i.e., an apparatus for generating hydrogen water), a nondestructive hydrogen adding apparatus (for example, an apparatus for non-destructively adding molecular hydrogen to the inside of a biocompatible liquid bag such as an infusion).

The hydrogen gas supplying apparatus enables to mix molecular hydrogen generated by the reaction of a hydrogen generating agent (for example, metal aluminum, magnesium hydride, and the like) and water, with a diluting gas (for example, air, oxygen, and the like) at a predetermined ratio (Japanese Patent No. 5228142, and the like). Alternatively, molecular hydrogen generated by the electrolysis of water is mixed with a diluting gas such as oxygen or air (Japanese Patent No. 5502973, Japanese Patent No. 5900688, and the like). The apparatus enables to prepare a molecular hydrogen-containing gas with a hydrogen concentration in the range of 0.5 to 18.5 vol %.

The hydrogen adding device is an apparatus that generates hydrogen using a hydrogen generating agent and a pH adjuster, and dissolves it in a biocompatible liquid such as water (Japanese Patent No. 4756102, Japanese Patent No. 4652479, Japanese Patent No. 4950352, Japanese Patent No. 6159462, Japanese Patent No. 6170605, JP 2017-104842 A, Japanese Patent No. 6159462, and the like). Examples of combinations of the hydrogen generating agent and the pH adjuster include metal magnesium and a strongly acidic ion exchange resin or an organic acid (for example, malic acid, citric acid, and the like), and metal aluminum powder and calcium hydroxide powder. The device enables to prepare a hydrogen-dissolved liquid with a dissolved hydrogen concentration of about 1 to 10 ppm (for example, trade name “7Water” (manufactured by Quasia), and the like).

A nondestructive hydrogen adding apparatus is an apparatus or device for adding hydrogen molecules to a commercially available biocompatible liquid such as an infusion (for example, sealed in a hydrogen-permeable plastic bag such as a polyethylene bag) from the outside of the package, and is commercially available from, for example, MiZ Company Limited (http://www.e-miz.co.jp/technology.html). This apparatus can aseptically dissolve hydrogen in a biocompatible liquid by immersing the bag containing the biocompatible liquid in saturated hydrogen water until the hydrogen permeates the bag and reaches concentration equilibrium. The apparatus is composed of, for example, an electrolysis tank and a water tank, and the water in the water tank circulates through the electrolysis tank and water tank, which apparatus enables to generate hydrogen by electrolysis. Alternatively, a simplified disposable device can be used for the same purpose (e.g., JP 2016-112562 A). This device contains a biocompatible liquid containing-plastic bag (a hydrogen-permeable bag, for example, a polyethylene bag) and a hydrogen generating agent (e.g., metal calcium, metal magnesium/cation exchange resin, and the like) in an aluminum bag, and the hydrogen generating agent is wrapped in, for example, a nonwoven fabric (e.g., a water vapor permeable nonwoven fabric). The hydrogen generated by wetting the hydrogen generating agent wrapped in the nonwoven fabric with a small amount of water such as water vapor permeates the plastic bag and is dissolved nondestructively and aseptically in the biocompatible liquid.

The molecular hydrogen-containing gas or molecular hydrogen-saturated biocompatible liquid (for example, water (e.g., purified water, sterilized water), physiological saline, an infusion, and the like), prepared using the above apparatuses or devices, may be administered orally or parenterally to humans having the above post-cerebral stroke sequelae.

Other forms of the composition of the present invention include dosage forms (for example, tablets, capsules, and the like) containing a hydrogen generating agent capable of generating hydrogen in the gastrointestinal tract and that is prepared for oral administration (or ingestion) to a human. It is preferable that the hydrogen generating agent be constituted by components approved as a food or food additive, for example.

As a method for administering the composition of the present invention to a human, pulmonary administration by inhalation, aspiration or the like is preferable when administering a molecular hydrogen-containing gas, and oral administration or intravenous administration (including infusion) is preferable when administering a molecular hydrogen-dissolved liquid. When inhaling a molecular hydrogen-containing gas, the molecular hydrogen-containing gas is inhaled through the mouth or nose via a nasal cannula or a mask-type device covering the mouth and nose, and then sent to the lungs to be delivered to the whole body through the blood.

With respect to the molecular hydrogen-dissolved liquid to be orally administered, a liquid preferably stored at a low temperature and cooled or a liquid stored at normal temperature may be administered to a human. Hydrogen is known to dissolve in water at a concentration of about 1.6 ppm (1.6 mg/L) at normal temperature and pressure, and the difference in solubility with temperature to be relatively small. Alternatively, when in the form of an infusion or an injection containing molecular hydrogen prepared using the above nondestructive hydrogen adding apparatus, for example, the molecular hydrogen-dissolved liquid may be administered to a human through a parenteral route of administration such as intravenous administration and intraarterial administration.

The molecular hydrogen-containing gas having the above hydrogen concentration or the hydrogen-dissolved liquid having the above dissolved hydrogen concentration can be administered to a human once or a plurality of times (for example, 2 to 3 times) per day, for a time period of 1 week to 3 months or more, for example, for 1 week to 6 months or more (for example, 1 year or more, 2 years or more, and the like). The molecular hydrogen-containing gas can be administered over a period of, for example, 30 minutes to 3 hours or more, preferably 1 to 3 hours or more, further preferably 1 to 2 hours per administration. Moreover, the molecular hydrogen-containing gas can be administered to a human by pulmonary administration through inhalation or aspiration under an atmospheric pressure environment or, for example, under a high atmospheric pressure environment (including the molecular hydrogen-containing gas) in a range exceeding standard atmospheric pressure (about 1.013 atm) and 7.0 atm or less, for example, 1.02 to 7.0 atm, preferably 1.02 to 5.0 atm, more preferably 1.02 to 4.0 atm, and further preferably 1.02 to 1.35 atm.

When treating a post-cerebral stroke sequela by the composition of the present invention, it is desirable to use a hydrogen gas generator, a hydrogen water generator, or a hydrogen gas adding apparatus (for example, an apparatus such as the above hydrogen gas supplying apparatus (or gaseous hydrogen inhaling (or breathing) apparatus), hydrogen adding equipment (i.e., a hydrogen water generator), or nondestructive hydrogen adding apparatus (i.e., an apparatus for nondestructively dissolving molecular hydrogen in a biocompatible liquid such as an infusion sealed in a hydrogen permeable bag), for which a sufficient therapeutic effect and safety has been confirmed.

### 2. Method for Ameliorating Post-Cerebral Stroke Sequela

The present invention further provides a method for ameliorating a post-cerebral stroke sequela in a human, which includes administering the composition containing molecular hydrogen as an active ingredient described in section 1 above to a human having a post-cerebral stroke sequela.

The composition containing molecular hydrogen, the cerebral stroke, the post-cerebral stroke sequelae, the dosage, the administration method, and the like are as described in section 1 above.

In the method of the present invention, the composition of the present invention can be administered to a human having a post-cerebral stroke sequela as early as possible after the onset of the cerebral stroke (for example, 1 to 7 days after onset), but the method of the present invention is also effective even when performed, for example, 10 days to 1 month or more (for example, 6 months or more, 1 year or more, 2 years or more, or 3 years or more) after onset.

In the method of the present invention, a gas (preferably air or oxygen) containing, for example, 0.5 to 18.5 vol % (preferably 5 to 10 vol %, 5 to 8 vol %, 6 to 8 vol %, 6 to 7 vol %, and the like, more preferably 5 to 8 vol %, such as 6 to 8 vol % and 6 to 7 vol %) of molecular hydrogen can be inhaled or aspirated by a human having a post-cerebral stroke sequela, for example, over 1 to 3 hours or more per day, and this can be continued, for example, for 1 to 3 months or more, 4 to 7 months or more, or 1 to 3 years or more.

Alternatively, in the method of the present invention, for example, 1 to 10 ppm (preferably 3 to 10 ppm, 4 to 10 ppm, 5 to 10 ppm, or the like) of molecular hydrogen-containing liquid may be administered to a human having a post-cerebral stroke sequela, for example, in an amount of 200 to 500 mL per administration in the case of intravenous administration, and 500 to 1000 mL per administration in the case of oral administration, and this can be continued, for example, for 0.5 to 3 months or more, 4 to 7 months or more, or 1 to 3 years or more.

In any of the administration forms, the administration period can be determined while monitoring the degree of improvement of the post-cerebral stroke sequela. For example, the administration period may be continued if signs of improvement of the sequela are observed, and the administration may be stopped if remission is observed.

The method of the present invention can further be used in combination with a treatment or rehabilitation for the symptoms of post-cerebral stroke sequelae (for example, numbness, convulsions, pain, language disorders, and the like). The combined use is expected to increase the effect of ameliorating the sequelae.

Examples of rehabilitation include exercise therapy for training to stand up, walk, or the like, occupational therapy for using chopsticks and training in housework, and speech therapy for speaking words and training how to swallow food.

### EXAMPLES

The present invention will be described more specifically with reference to the following Examples, but the technical scope of the present invention is not limited by the Examples.

### Example 1

### <Case 1 of Improvement of Cerebral Stroke Sequela Disorders by Inhalation of Molecular Hydrogen-Containing Air>

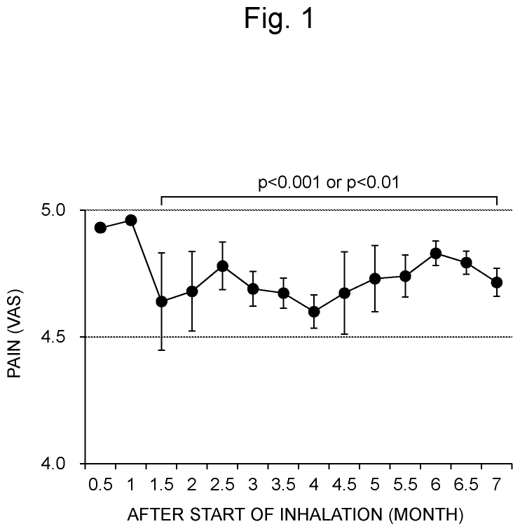

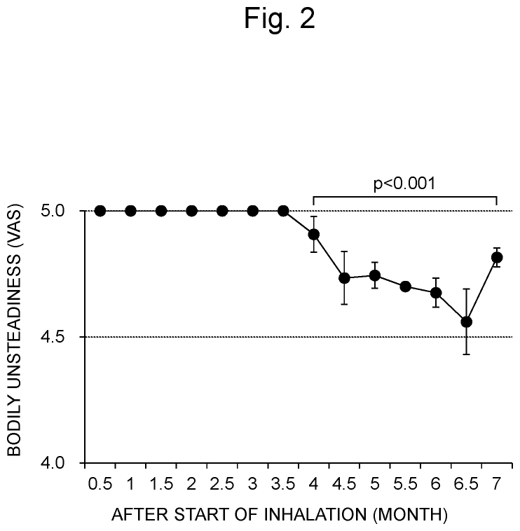

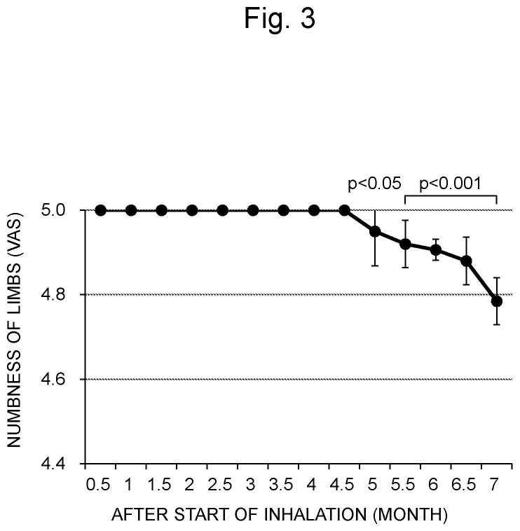

A woman in her 50s having developed cerebral hemorrhage (pontine hemorrhage) was unable to perform surgery due to the bleeding site being too deep within the pons, and was treated to stop the hemorrhage by drug administration. After about one year from the onset, inhalation (5 to 6 hours per day) of molecular hydrogen-containing air (apparatus manufactured by MiZ (MHG-2000α), molecular hydrogen concentration of about 6 to 7.5 vol %, 140 mL/min), as well as regular home-visit rehabilitation, home-visit massages and doctor visits were performed. The pain, bodily unsteadiness, and numbness of the limbs, which are sequela disorders, 0 to 7 months after inhalation of molecular hydrogen, were determined by calculating the mean±standard deviation of VAS values measured daily for 15 days, while analyzing the statistically significant difference of each measurement period relative to the start of inhalation of air containing molecular hydrogen (0.5 month) by Mann-Whitney U test, and as shown in FIGS. 1, 2 and 3, all symptoms showed a trend toward improvement from 4 months after inhalation. Specifically, the pain was around VAS 5.0 for one month from the start of inhalation, but then fluctuated between 4.5 and 4.8, indicating a significant trend toward improvement in data (FIG. 1). In addition, bodily unsteadiness improved to 4.5 to 4.8 from 3.5 months after the start of inhalation (FIG. 2), and furthermore, the numbness of the limbs was 5.0 until 4.5 months after the start of inhalation, but then improved to around 4.8 (FIG. 3). In particular, the central post-stroke pain that was the chief complain of the woman in this case, was a case of allodynia, a form of neuropathic pain (pain caused by stimuli that usually do not cause pain), and the results showing that the inhalation of air containing molecular hydrogen was effective on allodynia, which cannot be suppressed by modern medicaments, are noteworthy.

### Example 2

### <Case 2 of Improvement of Sequela Disorders of Cerebral Hemorrhage by Inhalation of Molecular Hydrogen-Containing Air>

A man in his 70s was hospitalized after developing cerebellar hemorrhage about eight years ago, then was transferred to a rehabilitation hospital after discharge to receive rehabilitation, which he is continuing to this day. When inhalation of molecular hydrogen-containing air (apparatus manufactured by MiZ (MHG-2000α), molecular hydrogen concentration of about 6 to 7.5 vol %, 140 mL/min) was started in October 2018, sequelae such as loss of motor control (no agility, inability to exercise, and inability to balance the body) were observed.

The inhalation of molecular hydrogen was performed daily for the first 2 months, 1 to 1.5 hours in the morning and 1 to 1.5 hours in the evening, then daily for 1 month, 2 to 3 hours in the morning and 2 to 3 hours in the evening, and finally daily for 1 month, 1 hour in the morning and 1 hour in the evening.

Approximately one month after inhaling molecular hydrogen, not only his previously clumsy articulation (language disorder) improved and the number of words easy to comprehend increased, but the man was now also able to walk in about 10 minutes what previously took him 20 minutes. Approximately two months after inhalation, he was able to talk while walking, his stride during rehabilitation lengthened, his ability to play Go, a game, increased, and he was able to handwrite New Year's cards. Approximately three months after inhalation, his speech became even more audible. Therefore, hydrogen inhalation was stopped at the hospital's discretion.

### INDUSTRIAL APPLICABILITY

Since the present invention can improve (or reduce) a post-cerebral stroke sequela in humans, combining it with the usually performed medical treatment or rehabilitation can support the daily life of a human having a post-cerebral stroke sequela.

All publications, patents and patent applications cited herein are incorporated herein by reference in their entirety.

1.-8. (canceled)

9. A method for ameliorating a post-cerebral stroke sequela in a human, comprising administering a composition comprising molecular hydrogen as an active ingredient to a human having a post-cerebral stroke sequela.

10. The method according to claim 9, wherein the cerebral stroke is selected from the group consisting of cerebral hemorrhage, subarachnoid hemorrhage, and cerebral infarction.

11. The method according to claim 9, wherein the sequela is at least one symptom selected from the group consisting of movement disorders, sensory disturbances, language disorders, central post-stroke pain, and allodynia.

12. The method according to claim 9, wherein the composition is a gas comprising an effective amount of molecular hydrogen and air or oxygen.

13. The method according to claim 12, wherein the composition is a gas comprising molecular hydrogen in an amount greater than zero (0 ) and not greater than 18.5 vol %.

14. The method according to claim 9, wherein the composition is a biocompatible liquid comprising an effective amount of molecular hydrogen.

15. The method according to claim 14, wherein the biocompatible liquid is water, physiological saline, or a transfusion.

16. The method according to claim 14, wherein a concentration of molecular hydrogen dissolved in the biocompatible liquid is 1 to 10 ppm.
